(19) United States
(12) Patent Application Publication
(10) Pub. No.: US 2006/0154247 A1
(43) Pub. Date: Jul. 13, 2006

(54) Methods,compositions and kits for cell separation

(76) Inventors: Matthew J Baker, Maidstone (GB); Matthew A Crow, Kent (GB)

Correspondence Address: Margaret J Sampson;Vinson & Elkins, 2300 First City Tower, 1001 Fannin Street, Houston, TX, 77002-6760, US

(21) Appl. No.: 10/516,204

(22) PCT Filed: Jun. 2, 2003
(86) PCT No.: PCT/GB03/02361

§ 371 Date: Nov. 23, 2005

(30) Foreign Application Priority Data
May 31, 2002 (GB) 0212825.4

(51) Int. Cl. C12Q 1/68; C12N 1/08

(52) U.S. Cl. 435/6; 435/270

(57) ABSTRACT

Methods, compositions and kits for concentrating or separating cells containing target nucleic acid are disclosed, especially m mixtures containing the cells and other components such as impurities. The methods can keep a large proportion of the cells intact, allowing the cells to be employed after separation (e.g. cultured) and/or which facilitates the recovery of nucleic acid from the cells. The method employs flocculating agents, such as polyamines or cationic detergents, to form complexes with cells causing them to aggregate and so separated from other components of the mixture. Conveniently, the separation of the aggregated cells can be effected with a solid phase which is capable of binding the cells, such as magnetic beads or filters.

### FIELD OF THE INVENTION

The present invention relates to methods, compositions and kits for cell separation, and in particular for separating cells from a mixture in which they are present with impurities, and more especially for use in methods which then allow the purification of target nucleic acid present in the cells.

### BACKGROUND OF THE INVENTION

The separation of cells from mixtures containing them and unwanted impurities is a challenging problem in the art. This is particularly the case where the cells are present in a culture broth, a biological sample or similar complex mixture as the methods employed need to capture a high proportion of the cells and capture substantially all of the cells intact, i.e. without killing or lysing the cells which would cause the release of cellular debris to further contaminate the mixture. This means that the reagents used in the cell concentration and separation steps must capture the cells very efficiently and from a range of cell densities, and not interfere by lysing the cell walls or making them “leaky” to nucleic acid before they are separated. Also, the reagents used should not interfere with downstream steps employing the cells, recovering nucleic acid from the cells and/or the processing of the nucleic acid, e.g. in carrying out PCR or other analytical techniques.

The separation of cells from cultures using flocculating agents such as polyethylenimine (PEI) is known in the art, see for example Kamath and D'Souza, Enzyme Microb. Technol., 13:935-939, 1991, which reports the capture of cells on cotton cloth coated with PEI. However, this paper is concerned with obtaining immobilised cells for use in bioreactors rather than the analytical processing of cells or the nucleic acid contained within them. Indeed, these prior art methods attempted to remove DNA from the cell cultures.

EP 0 515 484 A (Amersham International plc) discloses methods using magnetic beads formed from a magnetic material such as iron oxide, and optionally an organic polymer, for removing impurities such as cell debris, proteins and chromosomal DNA from a lysate mixture, thereby allowing the separation of a supernatant containing nucleic acid of interest. This application also discloses the use of the same type of beads for precipitating nucleic acid of interest from a supernatant and using the magnetic properties of the beads to draw down nucleic acid non-specifically binding to them. In passing, the application also refers to the precipitation of bacteria, tissue culture cells and blood cells using conventional precipitants, such as ethanolic sodium acetate at pH 5.2, and magnetic bead induced precipitate separation. However, the use of alcoholic precipitation in prior art methods suffers from the disadvantage that it causes cell death and lysis.

WO99/29703 and WO02/48164 (DNA Research Innovations Limited) disclose a wide range of ‘charge switch’ materials, typically in the form of solid phases, which are capable of binding nucleic acid present in a sample at a first pH and releasing the nucleic acid at a second, higher pH. These charge switch materials can be employed in the purification of nucleic acid from samples such as biological samples and lysis mixtures. The materials can be used in the form of magnetic beads or incorporated on the surface of pipettes or tubes.

U.S. Pat. No. 6,284,470 (Promega Corporation) discloses kits comprising two species of magnetic beads, a first which forms a complex with disrupted biological material present in a lysis mixture with a target nucleic acid and a second which forms a complex with the target nucleic acid under conditions which promote specific adsorption of the nucleic acid to the particles. The second species of magnetic particles may have charge switch properties, that is the binding of nucleic acid to the particles is pH dependent. This patent also describes the use of magnetic particles to concentrate or harvest cells such as bacteria or white blood cells by forming a complex between the cells and magnetic beads, e.g. derivatised with glycidyl-histidine.

There remains a need in the art for new methods of separating cells, and in particular for methods which are largely capable of avoiding cell lysis and which are readily susceptible to automation.

### SUMMARY OF THE INVENTION

Broadly, the present invention concerns methods, compositions and kits for concentrating or separating cells, especially from mixtures containing the cells and other components such as impurities. In preferred aspects, the present invention concerns a method of separating cells which is capable of keeping a large proportion of the cells intact and which therefore allows the cells to be employed after separation (e.g. cultured) and/or which facilitates the recovery of nucleic acid from the cells. The present invention is based on the finding that flocculating agents, such as polyamines or cationic detergents, form complexes with cells causing them to aggregate. For cells present in mixtures, the aggregation of the cells allows them to be readily separated from other components of the mixture. Conveniently, the separation of the aggregated cells can be effected with a solid phase which is capable of binding the cells, such as magnetic beads or filters.

Accordingly, in a first aspect, the present invention provides a method of separating cells present in a mixture with other materials, the method comprising:
contacting the mixture containing the cells with a flocculating agent capable of aggregating the cells, wherein the flocculating agent is a polyamine or a cationic detergent, and a solid phase capable of binding the cells; and,
separating the aggregated cells from the mixture using the solid phase.

The solid phase can be brought into contact with the cells before, after or simultaneously with the addition of the flocculating agent. In one embodiment, the flocculating agent is coupled to (preferably covalently linked to), mixed with or associated with the solid phase. This has the advantage of causing the cells to flocculate on the solid phase which can then be used to separate the cells from the mixture. In an alternative embodiment, the flocculating agent is initially soluble when added to the mixture containing the cells and forms an insoluble precipitate with the cells. In either case, the aggregation or precipitation of the cells may be enhanced using an agent which promotes or enhances this process as described below.

Examples of suitable solid phases for use in accordance with the present invention include magnetic beads, non magnetic beads, filters, filter columns, spin filter columns, membranes, particles, beads (e.g. silica beads) or frits, sinters, glass beads or slides, metal surfaces, fibres, polysaccharides or any plastic surface such as a tube, tip, probe or well. Magnetic beads are a particularly preferred solid phase, conveniently having average diameters between 0.1-20 μm. The solid phase may be in a soluble or insoluble form composed of inorganic or organic materials or composites thereof. By way of example, the solid phase may comprise materials such as plastics, glasses, polysaccharides, metal oxides, metal hydroxides/hydrates, salts, silicates, clays, lignins, charcoals and other insoluble fine particulates.

In the present invention, preferably a substantial proportion of the cells are captured intact. This means that the chemicals used must capture the cells efficiently, i.e. from a range of cell densities, and not interfere by killing or lysing the cell walls or making them “leaky” to nucleic acid before they are separated. In preferred embodiments, the present invention has the further advantage that the cells are viable after separation and can therefore be cultured or otherwise employed. Also, it is preferable that the reagents used are compatible with recovering the nucleic acid from the cells or inhibit downstream nucleic acid analysis, e.g. by PCR or other techniques.

Thus, in the context of the present invention, “not substantially lysed” in the cell separation step of the method means that less than 20%, more preferably less than 10%, more preferably less than 5%, more preferably less than 2% and most preferably less than 1% of the cells in the population treated according to the method are lysed. The extent of cell lysis can readily be determined, e.g. by counting lysed and non-lysed cells present in a sample under a microscope. As mentioned above, it is also preferably that a substantial proportion of the cells are viable after separation according to the present invention. Cell viability can be readily assessed by growing a sample of the separated cells on an appropriate growth medium and in this context, ‘a substantial proportion’ means at least 50% of the cells are viable, more preferably at least 75% of the cells, more preferably at least 85% of the cells and most preferably at least 95% of the cells are viable.

In the present invention, a flocculating agent which is a “polyamine” means a substance having more than one covalently linked units, each unit having one or more amine groups, e.g. primary, secondary, tertiary, quaternary, aromatic or heterocyclic amine groups, which are positively charged at the pH at which the material is used in the cell separation method. Preferred polyamines comprise a plurality of covalently linked units. The units forming the polyamine may be the same or different. In addition to the amine groups, the polyamines may be unsubstituted or substituted with one or more further functional groups, e.g. to modify their properties of facilitate coupling onto a solid phase. Preferred examples of polyamines include polyamino acids, polyallylamines, polyalkylimines such as polyethylenimine, polymerised biological buffers containing amine groups and polyglucoseamines. All of these classes of polyamine may be substituted or unsubstituted. Preferred polyamines, and especially polyallylamines, have molecular weights in the range of about 10 kDa to about 100 kDa, more preferably from about 50 kDa to about 80 kDa, and most preferably about 70 kDa. As mentioned above, preferred embodiments of the invention employ polyamines which are initially soluble and precipitate on forming complexes with the cells or the polyamine are coupled to, mixed with or associated with the solid phase.

In embodiments of the invention in which the polyamine is a polyamino acid, the linked amino acids forming the polyamino acid may be the same or different. Preferred examples include poly-lysines or poly-histidines. The amino acids used to form the polyamino acid may be D or L amino acids or a mixture of both.

In embodiments of the invention in which the polyamine is a polyallylamine or polyallylamine. HCl, the polyallylamine is preferably represented by the formula:
Poly(allylamine Hydrochloride): [—CH₂CH(CH₂NH₂.HCl)—]ₙ or
Poly(allylamine): [—CH₂CH (CH₂NH₂)—]ₙ
where n is at least 3 and the polyallylamine may be unsubstituted or have one or more further substitutions not shown in the simple formulae above. Such materials can be produced by the polymerisation of 2-propen-1-amine or a similar monomer comprising an alkene and an amine functional groups. Examples of polyallylamine can be supplied by Aldrich in the forms of solid of as solutions (e.g. 20 wt % solutions), both of which are usuable according to the present invention. Exemplary polyallylamines include poly(allylamine) reference 47,914-4 (20 wt % solution, Mw ca 65,000), poly(allylamine) reference 47,913-6 (20 wt % solution, Mw ca 17,000), poly(allylamine hydrochloride) reference 28,321-5 (solid, Mw ca 15,000) and poly(allylamine hydrochloride) reference 28,322-3 (solid, Mw ca70,000) all described in the 2001 Aldrich Catalogue, page 1385.

In embodiments of the invention in which the polyamine is a polyalkylimines such as polyethylimine (PEI), for example as represented by the formulae: polyethylenimine: (—NHCH₂CH₂—)ₓ[—N(CH₂CH₂NH₂)CH₂CH₂—]_{y}.

In embodiments of the invention in which the polyamine is a polymerised biological buffer such as poly Bis-Tris. Examples of biological buffers which have amine groups and can be polymerised and employed in the present invention include:
Bis-2-hydroxyethyliminotrishydroxymethylmethane (Bis-Tris), pKa 6.5.
1,3-bistrishydroxymethylmethylaminopropane (Bis-Tris propane), pKa 6.8.
N-trishydroxymethylmethylglycine (TRICINE), pKa 8.1.
Trishydroxymethylaminomethane (TRIS), pKa 8.1.

In embodiments of the invention in which the polyamine is a polyglucoseamine such as chitosan, a readily available material derived from the shells of crustacea and formed from repeating units of D-glucoseamine.

Other materials useful in flocculating cells are cationic detergents, such as hexamethidrine bromide, benzalkonium chloride, DTAB, CTAB, N-lauryl sarcosine ,cetrimide, polymyxins, or anti-septic or anti-microbial compounds.

In a further aspect, the present invention provides a composition comprising a solid phase and a flocculating agent, wherein the flocculating agent is a polyamine or a cationic detergent. As above, the flocculating agent may be associated with, mixed with or coupled to the solid phase. In embodiments in which the polyamine or detergent is coupled to the solid phase, covalently coupling is preferred.

In this aspect of the invention, the solid phase is preferably in the form of a bead, and more preferably a magnetic bead, for example having an average diameter between 0.1-20 μm. The solid phase may be formed from a material which is capable of binding nucleic acid at a first pH and releasing the bound nucleic acid at a second higher pH, i.e. a charge switch solid phase, for example as disclosed in WO02/48164 or WO99/29703. This means that one solid phase can be employed in the separation of cells from impurities and then in the subsequent purification of nucleic acid contained with the cells. This has advantages in simplifying the reagents needed to carry out such purification protocols and making them more susceptible to automation.

In a further aspect, the present invention provides a kit for separating cells from a mixture where the cells are present with impurities, the kit comprising:
a flocculating agent capable of aggregating the cells, wherein the flocculating agent is a polyamine or a cationic detergent;
a first solid phase which is capable of binding the aggregated cells;
optionally a second solid phase for purifying nucleic acid in the cells, the solid phase being capable of binding nucleic acid at a first pH and releasing the bound nucleic acid at a second higher pH (i.e. a charge switch solid phase, for example as disclosed in WO02/48164 or WO99/29703).

In preferred kits, the first and second solid phases may be the same, i.e. a charge switch solid phase can be employed to bind the cells and also in the purification of nucleic acid contained with the cells.

The present invention is widely applicable to many different types of samples containing cells including, but not limited to, culture broths, biological samples such as blood and tissue, foodstuffs, water contaminated liquids, host cells, e.g. separating cells such as Gram negative and Gram positive bacteria (e.g. E. coli), filamentous bacteria or fungi (such as Streptomyces), yeast cells, mammalian cells, plant cells and plant protoplasts.

In some preferred embodiments of the invention, the flocculating agent is used in conjunction with an agent to promote the aggregation of the cells. This agent may be a change in pH or temperature, a divalent or polyvalent ion, a change in counter ion to the flocculating agent, a cross-linking agent, a change-in concentration, evaporation. In a preferred embodiment of the invention, divalent or polyvalent anions are added to the mixture containing cells in order to promote flocculation. In a particularly preferred embodiment, phosphate ions are added. However, the phosphate ions may be substituted for any divalent or polyvalent anion including, but not limited to, sulphates and polycarboxylates. Without wishing to be bound by any particular theory, the inventors believe that when divalent cations such as phosphates are used, a polyelectrolyte complex is formed that becomes insoluble around the cells aiding the aggregation of cells and hence separation.

To carry out cell separation, the cell sample is brought in contact with the flocculating agent and solid phase. The cells associate with them, allowing the solid phase to be used to remove the complex from solution. Separation may be achieved by a range of well, known in the art such as vacuum filtration, syringe filtration, magnetic separation, electrophoresis, centrifugation, sedimentation or evaporation or liquid removal techniques.

After separation, the cells may be collected and cultured, stored for archive purposes or treated to release important biomolecules such as nucleic acids, proteins, metabolites, carbohydrates or lipid components or complexes thereof. Significant lysis of the cells during separation is avoided so that the biomolecules inside the cell are not lost. Thus, in a further embodiment, the methods of the present invention may comprise the step of culturing cells separated from the mixture.

The method of separating cells may be followed with steps to purify target biomolecules, and especially nucleic acid, contained within the cells. By way of example, the target nucleic acid may be non-genomic nucleic acid which is separated from genomic nucleic acid retained inside the cells. Non-genomic nucleic acid includes vectors, plasmids, self replicating satellite nucleic acid or cosmid DNA, or vector RNA. Other forms of target nucleic acids may include bacteriophages such as Lambda, M13 and viral nucleic acids. In a preferred embodiment, the non-genomic nucleic acid sample is plasmid DNA.

In preferred embodiments, the method is used to separate cells containing nucleic acid of interest, and the initial step of aggregating the cells may be part of a method of purifying the nucleic acid, as described in more detail below. Thus, in such embodiments of the invention, the method may comprise additional processing or purification steps carried out on the cell sample, for example involving one or more of the additional steps of:
(a) isolating the target nucleic acid; or
(b) analysing the target nucleic acid; or
(c) amplifying the target nucleic acid; or
(d) sequencing the target nucleic acid.

These steps are discussed in more detail below.

In a preferred embodiment, the invention may further comprise obtaining a sample of target nucleic acid from cells containing the target nucleic acid and genomic nucleic acid, the method comprising having separated the cells from culture broth, the further steps of:
suspending the cells in an aqueous medium which causes the target nucleic acid to leak from the cells into the aqueous medium; and
obtaining the sample of the nucleic acid from the aqueous medium;
wherein the cells are substantially not lysed during the above steps and substantially retain the genomic nucleic acid within the cells.

The details of this method are provided in PCT/GB02/005209. Preferably, this method does not substantially cause the release of cellular endotoxins, thereby allowing the separation of the target nucleic acid from the cellular endotoxins, in addition to genomic nucleic acid or RNA. In a preferred embodiment of this method, the target nucleic acids may be 100 kb or less, or more preferably 50 kb or less, or more preferably 20 kb or less or even more preferably 10 kb or less in size. The size of nucleic acids can be determined by those skilled in the art, e.g. using gel electrophoresis technique employing a polyacrylamide or agarose gel, e.g. see Ausubel et al, Short Protocols in Molecular Biology, John Wiley and Sons, NY, 1992.

Alternatively, the cells separated according to the above method may be lysed and target nucleic acid purified from the lysate, for example using a charge switch solid phase referred to above, a nucleic acid binding solid phase as described in EP 0 389 063 A in which silica or a derivative thereof is used to bind nucleic acid in the presence of a chaotrope.

In either case, the target nucleic acid, such as a plasmid, can be separated from the media containing the cells according to the present invention and the resulting aqueous media, i.e. the supernatant, used directly with out the requirement for further purification steps, e.g. for PCR or other analytical methods.

A range of techniques are available to the skilled person for purifying nucleic acid are known in the art. Examples of purification techniques include ion-exchange, electrophoresis, silica solid phase with chaotropic salt extraction, precipitation, flocculation, filtration, gel filtration, centrifugation, alcohol precipitation and/or the use of a charge switch material described in our copending applications WO97/29703 and WO02/48164 and other purification or separation methods well known in the art. In preferred embodiments, the target nucleic acid is purified using a charge switch material, e.g. present on a solid phase, a pipette tip, beads (especially magnetic beads), a porous membrane, a frit, a sinter, a probe or dipstick, a tube (PCR tube, Eppendorf tube) or a microarray.

The target nucleic acid may also be the subject of amplification, conveniently using the polymerase chain reaction. PCR techniques for the amplification of nucleic acid are described in U.S. Pat. No. 4,683,195. In general, such techniques require that sequence information from the ends of the target sequence is known to allow suitable forward and reverse oligonucleotide primers to be designed to be identical or similar to the polynucleotide sequence that is the target for the amplification. PCR comprises steps of denaturation of template nucleic acid (if double-stranded), annealing of primer to target, and polymerisation. The nucleic acid probed or used as template in the amplification reaction may be genomic DNA, cDNA or RNA. PCR can be used to amplify specific sequences from genomic DNA, specific RNA sequences and cDNA transcribed from mRNA, bacteriophage or plasmid sequences. References for the general use of PCR techniques include Mullis et al, Cold Spring Harbor Symp. Quant. Biol., 51:263, (1987), Ehrlich (ed), PCR Technology, Stockton Press, NY, 1989, Ehrlich et al, Science, 252:1643-1650, (1991), “PCR protocols; A Guide to Methods and Applications”, Eds. Innis et al, Academic Press, New York, (1990).

Embodiments of the present invention will now be described in more detail by way of example and not limitation.

### DETAILED DESCRIPTION

### EXAMPLE 1

### Polyamine Flocculation, Capturing Cells on a Filter and Purifying DNA Using Charge Switch Magnetic Beads

0.75 ml of an overnight culture of E. coli/pUC19 was mixed with 10 μl of 50 mg/ml poly(allylamine hydrochloride), Mw=70 kDa approx., in a 0.45 μm spin-filter column for 1 minute. The spin-filter column was then centrifuged at 13000 rpm for 1 minute to remove liquid without blocking the filter and the flow through was discarded. In the spin-filter column, the pellet was resuspended in 100 μl of 10 mM Tris-HCl (pH 8.5), 1 mM EDTA buffer containing 100 μg/ml RNaseA and left for 1 minute. The resuspended cells were then mixed with 100 μl of a 1% (w/v) SDS, 0.2 M NaOH lysis solution for 3 minutes, then a precipitation buffer (1.0 M potassium acetate, 0.66 M KCl, pH 4.0) was gently mixed in to precipitate cell debris. The spin-filter column was centrifuged again for 1 minute at 13000 rpm and the flow through was mixed with 20 μl of CST magnetic beads (25 mg/ml) and incubated at room temperature for 1 minute. Samples were applied to a magnet for 1 min and the supernatant was discarded. The beads were then washed twice with 100 μl of distilled water and then purified plasmid DNA was eluted from the beads into 50 μl of 10 mM Tris-HCl (pH8.5). Purified plasmid DNA was visualised by gel electrophoresis in a 1% agarose gel containing ethidium bromide.

### EXAMPLE 2

### Polyamine Flocculation, Capturing Cells on Charge Switch Magnetic Beads and Purifying DNA Using Charge Switch Magnetic Beads

1.0 ml of an overnight culture of E. coli/pUC19 was mixed with 30 μl of CST magnetic beads (25 mg/ml) premixed with 5 mg/ml poly(allylamine hydrochloride), Mw=70 kDa approx., in a 1.5 ml microcentrifuge tube for 1 minute. The sample was then applied to a magnet for 1 minute to harvest the magnetic beads and flocculated cells. The supernatant was discarded and the magnetic pellet was resuspended in 100 μl of 10 mM Tris-HCl (pH 8.5), 1 mM EDTA buffer containing 100 μg/ml RNaseA and left for 1 minute. The resuspended cells were then mixed with 100 μl of a 1% (w/v) SDS, 0.2 M NaOH lysis solution for 3 minutes, then a precipitation buffer (1.0 M potassium acetate, 0.66M KCl, pH 4.0) was gently mixed in to precipitate cell debris. Cell debris was removed by applying the sample to a magnet for 1 minute. The supernatant was then mixed with 20 μl of CST magnetic beads (25 mg/ml) and incubated at room temperature for 1 minute. Samples were applied to a magnet for 1 minute and the supernatant was discarded. The beads were then washed twice with 100 μl of distilled water and then purified plasmid DNA was eluted from the beads into 50 μl of 10 mM Tris-HCl (pH8.5). Purified plasmid DNA was visualised by gel electrophoresis in a 1% agarose electrophoresis gel containing ethidium bromide.

### EXAMPLE 3

### Polyamine Flocculation, Capturing Cells on Particles of Magnetite and Purifying DNA Using Charge Switch Magnetic Beads

As example 2, but using 50 μl of magnetite (50 mg/ml) premixed with 10 mg/ml poly(allylamine hydrochloride), Mw=70 kDa approx., instead of 30 μl of CST magnetic beads (25 mg/ml) premixed with 5 mg/ml poly(allylamine hydrochloride), Mw=70 kDa approx.

### EXAMPLE 4

As example 2, but using 10 mg/ml poly(allylamine hydrochloride), Mw=70 kDa approx., instead of 5 mg/ml poly(allylamine hydrochloride), Mw=70 kDa approx.

### EXAMPLE 5

As example 2, but using 10 mg/ml poly-L-lysine instead of 5 mg/ml poly(allylamine hydrochloride), Mw=70 kDa approx.

### EXAMPLE 6

As example 2, but using 10 mg/ml poly-DL-lysine instead of 5 mg/ml poly(allylamine hydrochloride), Mw=70 kDa approx.

### EXAMPLE 7

As example 2, but using 10 mg/ml poly-L-histidine instead of 5 mg/ml poly(allylamine hydrochloride), Mw=70 kDa approx.

### EXAMPLE 8

As example 2, but using 10 mg/ml poly(allylamine hydrochloride), Mw=15 kDa approx., instead of 5 mg/ml poly(allylamine hydrochloride), Mw=70 kDa approx.

### EXAMPLE 9

As example 2, but using 1 mg/ml poly(allylamine), Mw=17 kDa approx., instead of 5 mg/ml poly(allylamine hydrochloride), Mw=70 kDa approx.

### EXAMPLE 10

As example 2, but using 1 mg/ml poly(allylamine), Mw=65 kDa approx., instead of 5 mg/ml poly(allylamine hydrochloride), Mw=70 kDa approx.

### EXAMPLE 11

As example 2, but using 10 mg/ml poly(ethylenimine), instead of 5 mg/ml poly(allylamine hydrochloride), Mw=70 kDa approx.

### EXAMPLE 12

As example 2, but using 10 mg/ml polymyxin B (Sigma-Aldrich catalogue number P-1004), instead of 5 mg/ml poly(allylamine hydrochloride), Mw=70 kDa approx.

### EXAMPLE 13

As example 2, but using 10 mg/ml benzalkonium chloride, instead of 5 mg/ml poly(allylamine hydrochloride), Mw=70 kDa approx.

### EXAMPLE 14

As example 2, but using 10 mg/ml hexadecytrimethylammonium bromide (‘Cetrimide’, ‘CTAB’) instead of 5 mg/ml poly(allylamine hydrochloride), Mw=70 kDa approx.

### EXAMPLE 15

### Mammalian Cell Separation

Red blood cell (RBC) lysis solution=10 mM NH4HCO3, 0.1% Tween 20.

White blood cell (WBC) digestion buffer=1% SDS, 1 mM EDTA, 10 mM Tris HCl pH8

Genomic precipitation buffer=6 M ammonium acetate.

10 ml of sheep's blood was mixed with 30 ml of ‘RBC lysis solution’ and incubated at room temperature for 10 minutes. The sample was then centrifuged at 2000 rpm for 10 min and the supernatant was discarded and the cell pellet was resuspended in 10 ml of 50 mM phosphate buffer. A 500 μl aliquot of the cell suspension was then mixed with 30 μl of CST magnetic beads (25 mg/ml), premixed with 1 mg/ml poly(allylamine hydrochloride), Mw=70 kDa approx., and incubated for 2 minutes. The sample was then held against a magnet for 2 minutes and the cell-suspension was seen to be clear, indicating that the cells had been removed from suspension. The supernatant was discarded and the pellet was resuspended in 500 μl of ‘WBC digestion buffer’ and mixed by pipetting up and down for 1 minute. 150 μl of ‘Genomic precipitation buffer’ was then added and the mixture was vortexed for 20 seconds, the resulting precipitate was removed by applying the sample to a magnet for 2 minutes. 500 μl of the supernatant was then gently mixed with 500 μl of isopropanol and genomic DNA was seen to form a precipitate.

### EXAMPLE 16

As example 15, but using 1 mg/ml poly-L-lysine instead of 1 mg/ml poly(allylamine hydrochloride), Mw=70 kDa approx.

### EXAMPLE 17

As example 15, but using 1 mg/ml poly-DL-lysine instead of 1 mg/ml poly(allylamine hydrochloride), Mw=70 kDa approx.

### EXAMPLE 18

As example 15, but using 1 mg/ml poly-L-histidine instead of 1 mg/ml poly(allylamine hydrochloride), Mw=70 kDa approx.

### EXAMPLE 19

As example 15, but using 1 mg/ml poly(allylamine hydrochloride), Mw=15 kDa approx., instead of 1 mg/ml poly(allylamine hydrochloride), Mw=70 kDa approx.

### EXAMPLE 20

As example 15, but using 1 mg/ml poly(allylamine), Mw=17 kDa approx., instead of 1 mg/ml poly(allylamine hydrochloride), Mw=70 kDa approx.

### EXAMPLE 21

As example 15, but using 1 mg/ml poly(allylamine), Mw=65 kDa approx., instead of 1 mg/ml poly(allylamine hydrochloride), Mw=70 kDa approx.

### EXAMPLE 22

As example 15, but using 1 mg/ml poly(ethylenimine), instead of 1 mg/ml poly(allylamine hydrochloride), Mw=70 kDa approx.

### EXAMPLE 23

As example 15, but using 1 mg/ml polymyxin B (Sigma-Aldrich cat. No. P-1004), instead of 1 mg/ml poly(allylamine hydrochloride), Mw=70 kDa approx.

### EXAMPLE 24

As example 15, but using 1 mg/ml benzalkonium chloride, instead of 1 mg/ml poly(allylamine hydrochloride), Mw=70 kDa approx.

### EXAMPLE 25

As example 15, but using 1 mg/ml ‘Cetrimide’ (hexadecyltrimethylammonium bromide) instead of 1 mg/ml poly(allylamine hydrochloride), Mw=70 kDa approx.

### EXAMPLE 26

As example 15, but omitting 5 mg/ml poly(allylamine hydrochloride), Mw=70 kDa approx., and using only the poly-Tris coated magnetic beads

### EXAMPLE 27

200 μl of sheep's blood was mixed with 600 μl ‘RBC lysis solution’ and incubated at room temperature for 10 minutes. The sample was then mixed with 50 μl of CST magnetic beads (25 mg/ml), premixed with 1 mg/ml poly(allylamine hydrochloride), Mw=70 kDa approx., and incubated for 2 minutes. The sample was then applied to a magnet for 2 minutes and the supernatant was discarded. The magnetic pellet was resuspended in 200 μl of 10 mM NaOH and incubated at room temperature for 1 minute. The resuspended pellet was then held against a magnet for 2 min to remove magnetic particles. Extracted DNA was then visualised by gel electrophoresis in a 1% agarose gel containing ethidium bromide.

### EXAMPLE 28

As example 27, but using 1 mg/ml poly-DL-lysine instead of 1 mg/ml poly(allylamine hydrochloride), Mw=70 kDa approx.

### EXAMPLE 29

As example 27, but using 1 mg/ml poly-L-histidine instead of 1 mg/ml poly(allylamine hydrochloride), Mw=70 kDa approx.

### EXAMPLE 30

As example 27, but using 1 mg/ml poly(allylamine hydrochloride), Mw=15 kDa approx., instead of 1 mg/ml poly(allylamine hydrochloride), Mw=70 kDa approx.

### EXAMPLE 31

As example 27, but using 1 mg/ml poly(allylamine), Mw=17 kDa approx., instead of 1 mg/ml poly(allylamine hydrochloride), Mw=70 kDa approx.

### EXAMPLE 32

As example 27, but using 1 mg/ml poly(allylamine), Mw=65 kDa approx., instead of 1 mg/ml poly(allylamine hydrochloride), Mw=70 kDa approx.

### EXAMPLE 33

As example 27, but using 1 mg/ml poly(ethylenimine), instead of 1 mg/ml poly(allylamine hydrochloride), Mw=70 kDa approx.

### EXAMPLE 34

As example 27, but using 1 mg/ml polymyxin B (Sigma-Aldrich catalogue number P-1004), instead of 1 mg/ml poly(allylamine hydrochloride), Mw=70 kDa approx.

### EXAMPLE 35

As example 27, but using 1 mg/ml benzalkonium chloride, instead of 1 mg/ml poly(allylamine hydrochloride), Mw =70 kDa approx.

### EXAMPLE 36

As example 27, but using 1 mg/ml ‘Cetrimide’ (hexadecytrimethylammonium bromide) instead of 1 mg/ml poly(allylamine hydrochloride), Mw=70 kDa approx.

### EXAMPLE 37

As example 27, but omitting 5 mg/ml poly(allylamine hydrochloride), Mw=70 kDa approx. and using only the poly-Tris coated magnetic beads.

### EXAMPLE 38

200 μl of sheep's blood was mixed with 600 μl ‘RBC lysis solution’ and incubated at room temperature for 10 minutes. The sample was then mixed with 50 μl of CST magnetic beads (25 mg/ml), premixed with 1 mg/ml poly(allylamine), Mw=65 kDa approx., and incubated for 2 minutes. The sample was then applied to a magnet for 2 min and the supernatant was discarded. The magnetic pellet was resuspended in 500 μl of ‘WBC digestion buffer’ and mixed by pipetting for 1 minute. 150 μl of ‘Genomic precipitation buffer’ was added and vortexed for 20 seconds to mix then the tube was placed against a magnet for 2 minutes. 500 μl of the supernatant was removed and mixed with 500 μl of isopropanol to precipitate any DNA. The sample was then incubated at −20° C. for 2 min followed by centrifugation at 13000 rpm for 10 minutes. The supernatant was discarded and the pellet was washed once with 500 μl of 70% (v/v) ethanol. The pellet was air-dried and then dissolved overnight in 10 mM Tris-HCl. The purified genomic DNA was then visualised by gel electrophoresis in a 1% agarose gel containing ethidium bromide.

### EXAMPLE 39

As example 38, but using 1 mg/ml poly-L-lysine instead of 1 mg/ml poly(allylamine hydrochloride), Mw=70 kDa approx.

### EXAMPLE 40

As example 38, but using 1 mg/ml poly-DL-lysine instead of 1 mg/ml poly(allylamine hydrochloride), Mw=70 kDa approx.

### EXAMPLE 41

As example 38, but using 1 mg/ml poly-L-histidine instead of 1 mg/ml poly(allylamine hydrochloride), Mw=70 kDa approx.

### EXAMPLE 42

As example 38, but using 1 mg/ml poly(allylamine hydrochloride), Mw=15 kDa approx., instead of 1 mg/ml poly(allylamine hydrochloride), Mw=70 kDa approx.

### EXAMPLE 43

As example 38, but using 1 mg/ml poly(allylamine), Mw=17 kDa approx., instead of 1 mg/ml poly(allylamine hydrochloride), Mw=70 kDa approx.

### EXAMPLE 44

As example 38, but using 1 mg/ml poly(allylamine), Mw=65 kDa approx., instead of 1 mg/ml poly(allylamine hydrochloride), Mw=70 kDa approx.

### EXAMPLE 45

As example 38, but using 1 mg/ml poly(ethylenimine), instead of 1 mg/ml poly(allylamine hydrochloride), Mw=70 kDa approx.

### EXAMPLE 46

As example 38, but using 1 mg/ml polymyxin B (Sigma Aldrich catalogue number P-1004), instead of 1 mg/ml poly(allylamine hydrochloride), Mw=70 kDa approx.

### EXAMPLE 47

As example 38, but using 1 mg/ml benzalkonium chloride, instead of 1 mg/ml poly(allylamine hydrochloride), Mw=70 kDa approx.

### EXAMPLE 48

As example 38, but using 1 mg/ml ‘Cetrimide’ (hexadecytrimethylammonium bromide) instead of 1 mg/ml poly(allylamine hydrochloride), Mw=70 kDa approx.

### EXAMPLE 49

As example 38, but omitting 5 mg/ml poly(allylamine hydrochloride), Mw=70 kDa approx. and using only the poly-Tris coated magnetic beads

### EXAMPLE 50

### Cells Separated Using the Present Invention can be Cultured

1 ml of overnight culture of E. coli/pUC19 was mixed with 30 μl of 50 mg/ml poly(allylamine hydrochloride), Mw=70 kDa approx. The resulting flock formed from the precipitation reaction was removed from the broth with a sterile inoculation loop and streaked out on to LBA plates containing 50 μg/ml ampicillin (to select for the β-lactamase gene on the pUC19 plasmid). Plates were incubated overnight at 37° C. Good bacterial growth was seen, indicating that the flocculation reaction did not kill the bacteria.

### EXAMPLE 51

1 ml of overnight culture of E. coli/pUC19 was mixed with 30 μl of CST beads premixed with 5 mg/ml poly(allylamine hydrochloride), Mw=70 kDa approx. The resulting magnetic precipitate was harvested by holding the tube against a magnet for 1 minute and discarding the supernatant. The pellet was then streaked on to LBA plates containing 50 μg/ml ampicillin (to select for the β-lactamase gene on the pUC19 plasmid) using a sterile inoculation loop. Plates were then incubated overnight at 37° C. Good bacterial growth was seen, indicating that the flocculation reaction did not kill the bacteria.

### EXAMPLE 52

Plasmid DNA purified using the method described in example 2 can be digested using restriction endonucleases (such as HindIII), showing that DNA can be used in molecular biological applications.

### EXAMPLE 53

1.0 ml of an overnight culture of E. coli/pUC19 was mixed with 50 μl of magnetite (50 mg/ml) premixed with 1 mg/ml Chitosan in a 1.5 ml microcentrifuge tube for 1 minute. The sample was then applied to a magnet for 1 minute to harvest the magnetic beads and flocculated cells. The supernatant was discarded and the magnetic pellet was resuspended in 100 μl of 10 mM Tris-HCl (pH 8.5), 1 mM EDTA buffer containing 100 μg/ml RNaseA and left for 1 minute. The resuspended cells were then mixed with 100 μl of a 1% (w/v) SDS, 0.2M NaOH lysis solution for 3 minutes, then a precipitation buffer (1.0M potassium acetate, 0.66M KCl, pH 4.0) was gently mixed in to precipitate cell debris. Cell debris was removed by applying the sample to a magnet for 1 minute. The supernatant was then mixed with 20 μl of CST magnetic beads (25 mg/ml) and incubated at room temperature for 1 minute. Samples were applied to a magnet for 1 minute and the supernatant was discarded. The beads were then washed twice with 100 μl of distilled water and then purified plasmid DNA was eluted from the beads into 50 μl of 10 mM Tris-HCl (pH8.5). Purified plasmid DNA was visualised by gel electrophoresis in a 1% agarose electrophoresis gel containing ethidium bromide.

### EXAMPLE 54

### Purification of Yeast Vectors

An overnight culture of yeast YPH501 containing vector ESC-Leu was prepared and 1 ml was mixed with 30 μl of magnetic beads adsorbed with polyamine. After the cells were separated with a magnet the supernatant was removed and the cells resuspended in a standard spheroplasting solution containing sorbital, mercaptoethanol and lyticase for 30 minutes. The spheroplasts were then lysed with 300 ul of 0.2M NaOH with 1% SDS which was then cleared by adding 30 ul of a 1.5M potassium acetate buffer pH4. Removal of the cellular debris was achieved by using the magnetic beads still present in the mixture to bind to the debris and separate with a magnet.

The references herein all expressly incorporated by reference.

1. A method of separating cells containing target nucleic acid, the cells being present in a mixture with other materials, and of purifying the target nucleic acid from the cells, the method comprising:
(a) contacting the mixture containing the cells with a flocculating agent capable of aggregating the cells, wherein the flocculating agent is a polyamine or a cationic detergent, and a solid phase capable of binding the cells;
(b) separating the aggregated cells from the mixture, using the solid phase; and
(c) purifying the target nucleic acid from the cells.

2. The method of claim 1, wherein the solid phase is brought into contact with the cells before, after or simultaneously with the addition of the flocculating agent.

3. The method of claim 1 or claim 2, wherein the cells are not substantially lysed after the separation step.

4. The method of any one of claims 1 to 3, wherein the cells are viable after the separation step.

5. The method of any one of the preceding claims, wherein the flocculating agent is coupled to, mixed with or associated with the solid phase causing the cells to flocculate on the solid phase which can then be used to remove the cells from the mixture.

6. The method of any one of claims 1 to 5, wherein the flocculating agent is initially soluble and forms a precipitate with the cells in the mixture.

7. The method of any one of the preceding claims, wherein the solid phase comprises magnetic beads, non magnetic beads, filters, membranes, particles, silica beads or frits, sinters, glass, polysaccharides or any plastic surface such as a tube, tip, probe or well.

8. The method of claim 7, wherein the solid phase is magnetic beads.

9. The method of any one of the preceding claims, wherein the solid phase is capable of binding nucleic acid at a first pH and releasing nucleic acid at a second, higher pH.

10. The method of any one of the preceding claims, further comprising adding divalent or polyvalent anions to the mixture to promote flocculation of the cells.

11. The method of claim 10, wherein the divalent or polyvalent anions are phosphate or sulphate ions.

12. The method of claim 10 or claim 11, wherein the anions are added before or after the flocculating agent.

13. The method of any one of the preceding claims, wherein flocculating agent is a polyamine.

14. The method of claim 13, wherein the polyamine is a polyamino acid, a polyallylamine, a polyalkylimine, a polyethylimine, a polymerised biological buffer containing amine groups, or a polyglucoseamine.

15. The method of any one of claims 1 to 12, wherein flocculating agent is a cationic detergent

16. The method of claim 15, wherein the cationic detergent is hexamethidrine bromide, benzalkonium chloride, DTAB, CTAB, N-lauryl sarcosine, cetrimide, polymyxins, or an anti-septic or anti-microbial compound.

17. The method of any one of the preceding claims, wherein the cells are present in. a culture broth or a biological sample.

18. The method of any one of the preceding claims, further comprising culturing the cells after separation from the mixture.

19. The method of any one of the preceding claims, wherein after step (b) the cells are lysed.

20. The method of claim 19, further comprising, after the step of lysing the cells, the step of binding cell debris to the solid phase and separating the cell debris and solid phase to provide a solution of target nucleic acid.

21. The method of claim 20, further comprising separating the nucleic acid from the solution.

22. The method of claim 21, wherein the nucleic acid is separated using a solid phase comprising silica or a derivative thereof to bind the nucleic acid.

23. The method of claim 21, wherein the nucleic acid is separated by contacting the solution of target nucleic acid with a solid phase is capable of binding nucleic acid at a first pH and releasing nucleic acid at a second, higher pH so that the nucleic acid binds to the solid phase.

24. The method of claim 23, further comprising changing the pH of the solution to the second, higher pH to release the target nucleic acid.

25. The method of any one of the preceding claims, further comprising analysing and/or amplifying and/or sequencing the target nucleic acid.

26. The method of any one of claims 1 to 18, further comprising:
obtaining a sample of target nucleic acid from cells containing the target nucleic acid and genomic nucleic acid, the method comprising having separated the cells from culture broth, the further steps of:
suspending the cells in an aqueous medium which causes the target nucleic acid to leak from the cells into the aqueous medium; and
obtaining the sample of the nucleic acid from the aqueous medium;
wherein the cells are substantially not lysed during the above steps and substantially retain the genomic nucleic acid within the cells.

27. A composition comprising a solid phase mixed with a flocculating agent, wherein:
(a) the flocculating agent is a polyamine or a cationic detergent; and
(b) the solid phase is a magnetic bead or the solid phase is formed from a material which is capable of binding nucleic acid at a first pH and releasing the bound nucleic acid at a second higher pH.

28. A kit for separating cells from a mixture where the cells are present with impurities and purifying nucleic acid present in the cell, the kit comprising:
a flocculating agent capable of aggregating the cells, wherein the flocculating agent is a polyamine or a cationic detergent;
a first solid phase which is capable of binding the aggregated cells; and
a second solid phase for purifying nucleic acid in the cells, the solid phase being capable of binding nucleic acid at a first pH and releasing the bound nucleic acid at a second higher pH.

29. The kit of claim 28, wherein the first and second solid phases are the same.

30. The kit of claim 28 or claim 29, wherein the first and/or the second solid phases are beads.

31. The kit of claim 30, wherein the bead is a magnetic bead.

32. The kit of any one of claims 28 to 31, wherein the solid phase is formed from a material which is capable of binding nucleic acid at a first pH and releasing the bound nucleic acid at a second higher pH.
